(19) United States
(12) Patent Application Publication
(10) Pub. No.: US 2019/0269535 A1
(43) Pub. Date: Sep. 5, 2019

(54) CENTRAL AIRWAY STENT REMOVAL DEVICE AND RELATED SYSTEMS AND METHODS

(71) Applicant: Regents of the University of Minnesota, Minneapolis, MN (US)

(72) Inventors: Felix Landaeta, Minneapolis, MN (US); Roy Joseph Cho, Minneapolis, MN (US); Douglas A. Devens, Minneapolis, MN (US)

(21) Appl. No.: 16/293,247

(22) Filed: Mar. 5, 2019

Related U.S. Application Data
(60) Provisional application No. 62/638,598, filed on Mar. 5, 2018.

(51) Int. Cl. A61F 2/95; A61B 1/00

(52) U.S. Cl. CPC A61F 2/95; A61B 1/267; A61F 2002/9528; A61B 1/0008

(57) ABSTRACT

A central airway stent removal device for use under direct visual observation with a flexible or ridged. The device includes an elongated shaft defining a central lumen configured to receive the endoscope and a hook mechanism attached to a distal end of the elongated shaft. The hook mechanism including a receiving platform extending away from the elongated shaft and a hook element at the distal end of the hook mechanism, the hook element defining a receiving slot having an entrance that opens towards the elongated shaft, in which the receiving slot is configured to receive an end of a stent intended to be removed from a body of a patient.

### CROSS-REFERENCE TO RELATED APPLICATION

This application claims priority from U.S. provisional patent application Ser. No. 62/638,598, filed Mar. 5, 2018, entitled the same, which is incorporated herein by reference.

### TECHNICAL FIELD

This disclosure relates to stent removal devices, and more particularly to central airway stent removal devices for use in placement, repositioning, or removal of central airway stents.

### BACKGROUND

Central airway stent procedures are used to relieve airway obstruction and stenosis caused by constrictions that are not suitable for airway resection or airway reconstruction. In particular, central airway stent procedures are an important option for the treatment of central airway stenosis arising from complication in lung transplant and lung cancer.

While central airway stent procedures provide a valuable option for addressing central airway obstruction and stenosis, complications can occur in both placement and removal of central airway stents. Example complications include stent migration away from a desired placement position, unwanted tissue growth within and around the stent, infection, necrosis, and the like. Many of these complications, as well as other indicators, can require removal of the central airway stent.

Conventionally, removal of a central airway stent required the use of standard surgical forceps. These forceps generally do not optimally couple with central airway stent mechanical properties and geometries as the forceps are not central-airway-stent specific. Because of the lack of non-specific surgical forceps within the industry, it is common during central airway stent removal procedures for the surgeon to spend long periods of time inefficiently capturing, destroying, and/or disassembling the central airway stent in order to remove it. Though some stents include mechanisms to aid in their removal (e.g., a string at an end of the stent that, when grasped and pulled by a forceps, causes the end of the stent to collapse to make the stent easier to remove), these mechanisms can fail, causing the surgeon to remove the stent in an alternative manner. Inefficient central airway stent removal can extended surgery times, increased healing time, or, require an aborted removal of the stent.

### SUMMARY

The examples disclosed herein relate to central airway stent removal devices and, in some examples, improved central airway stents for use with central airway stent removal devices. Placement, repositioning, or removal of central airway stents with the central airway stent removal devices discussed herein can be accomplished with either a flexible or rigid bronchoscopy, with the central airway stent under direct visual observation via an endoscope (e.g., bronchoscope) by a clinician during the procedure. The central airway stent removal devices may include the use of one or more of pneumatic, mechanical, magnetic, or thermal forces to remove central airway stents as discussed further below.

In some examples, the disclosure describes a central airway stent removal device for use under direct visual observation with a flexible or ridged, the device including an elongated shaft defining a central lumen configured to receive the endoscope and a hook mechanism attached to a distal end of the elongated shaft. The hook mechanism including a receiving platform extending away from the elongated shaft and a hook element at the distal end of the hook mechanism, the hook element defining a receiving slot having an entrance that opens towards the elongated shaft, in which the receiving slot is configured to receive an end of a stent intended to be removed from a body of a patient.

In some examples, the disclosure describes a process for removing a central airway stent, the process including receiving an endoscope within a central lumen of a stent removal device, in which the stent removal device includes an elongated shaft defining the central lumen configured to receive the endoscope and a hook mechanism attached to a distal end of the elongated shaft, in which the hook mechanism includes a receiving platform extending away from the elongated shaft, and a hook element at the distal end of the hook mechanism, the hook element also defining a receiving slot having an entrance that opens towards the elongated shaft. The method may further include advancing the hook element into a body of a patient and past a central airway stent positioned within the body of the patient, receiving a distal end of the central airway stent into the receiving slot such that a sidewall of the central airway stent is positioned adjacent to the receiving platform, rotating the hook element relative to the central airway stent to cause the central airway stent to at least partially collapse inward; and removing the central airway stent and the hook mechanism by proximally withdrawing the elongated shaft from the body of the patient.

In some examples, the disclosure describes a central airway stent removal device for use under direct visual observation with an endoscope, the device including a ridged or flexible elongated shaft including an inner lumen arranged coaxially within the shaft, the inner lumen including a control wire, a hook mechanism coupled to a distal end of the shaft, the hook mechanism including an inner support element including a hook detent arranged at a distal portion of the inner support element, the inner support element coupled to a distal end of the control wire, the inner support element and control wire configured to selectively and slidably move within the inner channel of the shaft via manipulation of the control wire, a receiving platform extending away from the elongated shaft, and a hook element at the distal end of the hook mechanism, the hook element defining a receiving slot having an entrance that opens towards the elongated shaft. The receiving slot is configured to receive an end of a stent and the hook detent is configured to selectively engage with the hook element to capture the stent between the receiving platform and the inner support element.

In some examples the disclosure describes a central airway stent removal device for use under direct visual observation with an endoscope, the device comprising: a shaft; and a head coupled with the shaft, at least a portion of the head configured to frictionally engage with an internal surface of a central airway stent such that advancing or retracting the shaft causes the central airway stent to advance or retract within a central airway.

In some examples, the head is a pneumatic head coupled to a distal end of the shaft, the pneumatic head being in fluidic engagement with the aperture, and wherein the head further comprises one or more bellows fittings arranged on the pneumatic head and configured to engage with the inner surface of the central airway stent.

In another example, the head further comprises a collet coupled to a distal end of the shaft and further configured to frictionally engage with the inner surface of the central airway stent; and the device further comprises a separator slidably coupled to the shaft, wherein the separator is arranged coaxially with the shaft and the collet and is configured to coaxially engage an outer surface of the central airway stent.

In yet another example, the head comprises at least one magnetic element configured to magnetically engage with a portion of the inner surface of the central airway stent.

In still another example, at least a portion of the head comprises a shape memory material configured to expand and engage with the central airway stent under predetermined thermal conditions.

In another example, a central airway stent removal device for use under direct visual observation with an endoscope, the device comprising: a shaft including an inner channel arranged coaxially within the shaft, the inner channel including a control wire; an inner support element including a hook detent arranged at a distal portion of the inner support element, the inner support element coupled to a distal end of the control wire, the inner support element and control wire configured to selectively and slidably move within the inner channel of the shaft via manipulation of the control wire; and a hook element fixedly coupled to a distal end of the shaft, wherein the hook element is configured to selectively engage with an edge of a wall of a central airway stent along an exterior surface of the central airway stent and the hook detent is configured to selectively engage with the hook element from along an interior surface of the central airway, such that the wall of a central airway stent is enclosed between the hook element and the inner support element to move central airway stent.

The above summary is not intended to describe each illustrated example or every implementation of the subject matter hereof. The figures and the detailed description that follow more particularly exemplify various examples.

### BRIEF DESCRIPTION OF THE DRAWINGS

The subject matter may be more completely understood in consideration of the following detailed description of various examples in connection with the accompanying figures, in which:

FIG. 1 is a perspective view of an example pneumatic central airway stent removal device.

FIG. 2 is a perspective view of an example pneumatic central airway stent removal device.

FIG. 3 is a perspective view of an example mechanical central airway stent removal device.

FIGS. 4A-4E are perspective views of various stages of a removal process where the central airway stent removal device of FIG. 3 removes a central airway stent from a central airway.

FIG. 5 is a side view of an example magnetic central airway stent removal device.

FIG. 6 is a side view of an example thermally activated central airway stent removal device.

FIG. 7 is a flowchart showing the use of a pneumatic central airway stent removal device in removing a central airway stent with the aid of an endoscope.

FIG. 8A is a perspective view of an example hook-capture central airway stent removal device.

FIG. 8B is a perspective view of a distal tip of the hook-capture central airway stent removal device of FIG. 8A.

FIG. 8C is a perspective view of a distal tip of the hook-capture central airway stent removal device of FIG. 8A.

FIG. 8D is a perspective view of the hook-capture central airway stent removal device of FIG. 8A engaged with a central airway stent according to an example.

FIG. 9 is a flowchart showing the use of a hook-capture central airway stent removal device in removing a central airway stent with the aid of an endoscope.

FIG. 10A is a perspective view of another example hook-capture central airway stent removal device.

FIG. 10B is a side view of the hub assembly of the central airway stent removal device of FIG. 10A.

FIGS. 10C and 10D are perspective views of the hook mechanism of the central airway stent removal device of FIG. 10A.

FIG. 10E is side view of the hook element of the central airway stent removal device of FIG. 10A.

FIG. 10F is perspective view of the hook mechanism from the perspective of an endoscope received by the central airway stent removal device of FIG. 10A.

FIG. 11A is a perspective view of another example hook-capture central airway stent removal device according to an example.

FIGS. 11B and 11C are perspective and side views of the hub assembly of the central airway stent removal device of FIG. 11A with a portion of the grip removed.

FIGS. 11D and 11E are perspective views of the hook mechanism of the central airway stent removal device of FIG. 11A.

FIG. 11F is a perspective view of the central airway stent removal device of FIG. 11A and an endoscope that can be coupled to the device.

FIG. 11G is perspective view of the hook mechanism from the perspective of an endoscope received by the central airway stent removal device of FIG. 11A.

FIG. 12 is a flow diagram illustrating an example technique for removing a central airway stent using the central airway stent removal device described herein.

FIGS. 13A to 14B are side views of the hook element of the central airway stent removal device of FIG. 11A engaging with a stent according to the techniques described by FIG. 12.

While various examples are amenable to various modifications and alternative forms, specifics thereof have been shown by way of example in the drawings and will be described in detail. It should be understood, however, that the intention is not to limit the claimed inventions to the particular examples described. On the contrary, the intention is to cover all modifications, equivalents, and alternatives falling within the spirit and scope of the subject matter as defined by the claims.

### DETAILED DESCRIPTION OF THE DRAWINGS

Examples disclosed herein relate to central airway stent removal devices and, in some examples, improved central airway stents for use with central airway stent removal devices. Placement, repositioning, or removal of central airway stents with the central airway stent removal devices discussed herein can be accomplished with either a flexible or rigid bronchoscopy, with the central airway stent under direct visual observation by a clinician via an endoscope during the procedure.

As used herein, an endoscope refers to an optical device used by a clinician to used to look deep into the body of a patient. Endoscopes may be include, but are not limited to bronchoscopes, telescopes, borescopes, and the like. An endoscope may include either a rigid or flexible shaft and can include a camera apparatus to provide a digital display of the treatment area or may include a viewing port for the clinician to visually observe the treatment area. Endoscopes are commercially available and come in standard lengths (e.g., 40 cm to 70 cm) and diameters (e.g., 3.5 mm to 8.5 mm). Endoscopes often include a light source or a port to couple to a light source in order to illuminate the surgical field. The central airway stent removal devices described herein may be used in conjunction with an endoscope and in some examples may even be configured to receive and couple to an endoscope.

Central airway stents can be configured for use in various portions of the central airway, e.g., in or near the trachea, bronchi, or larynx, etc., and for use in a variety of patients (e.g., pediatric, adult, etc.). Central airway stents can have diameters in a range from about 5 millimeters (mm) to about 30 mm and lengths typically set to 10 mm, 40 mm, 60 mm, 80 mm, or 120 mm. The central airway stent removal devices may be configured to be compatible with central airway stents in these ranges of sizes. Further, central airway stent removal devices as discussed herein can be used for repositioning of a central airway stent, as well as its original placement or subsequent removal.

As described in further detail below, the central airway stent removal devices may use vacuum, friction, mechanical (e.g., hook or clasp), or magnetic force to hold the central airway stent in order to facilitate placement, repositioning, or removal of the central airway stent. Central airway stent removal devices can optionally use a separator to assist in removing central airway stents, especially in central airway stents that are particularly fixated to the central airway wall. The separator, as described below, can be configured to slide around the exterior of the central airway stent (once the central airway stent is held by the aforementioned forces) to sever the tissue connection between the central airway stent and the central airway wall to facilitate the stent removal. Further, central airway stent removal devices are used with an endoscope (e.g., bronchoscope) such that the central airway stent procedures are conducted under direct visual observation by a clinician via the endoscope, in contrast with procedures related to stents used outside of the airway, in other parts of the body.

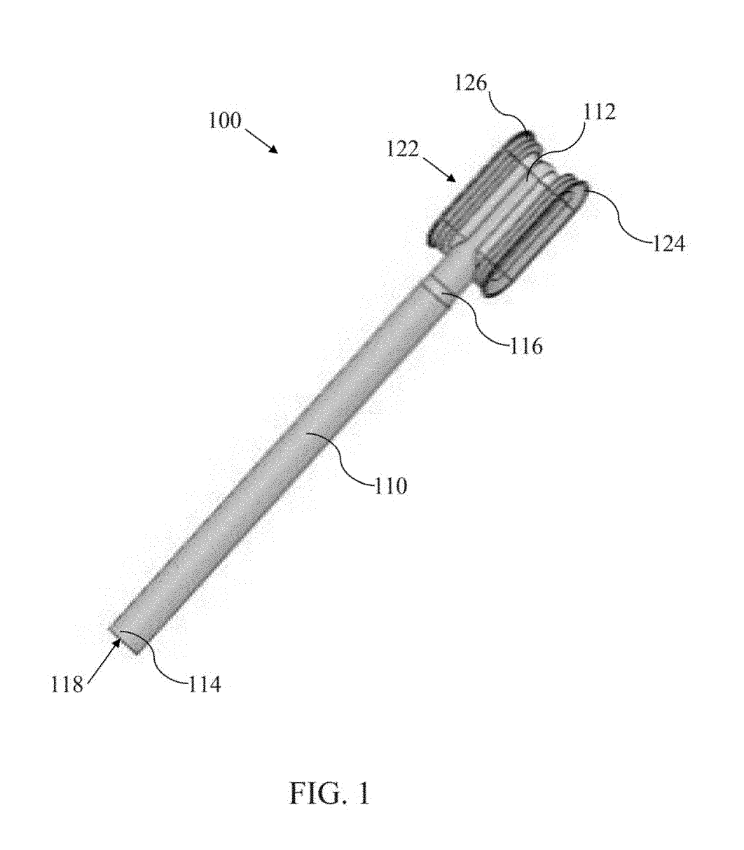

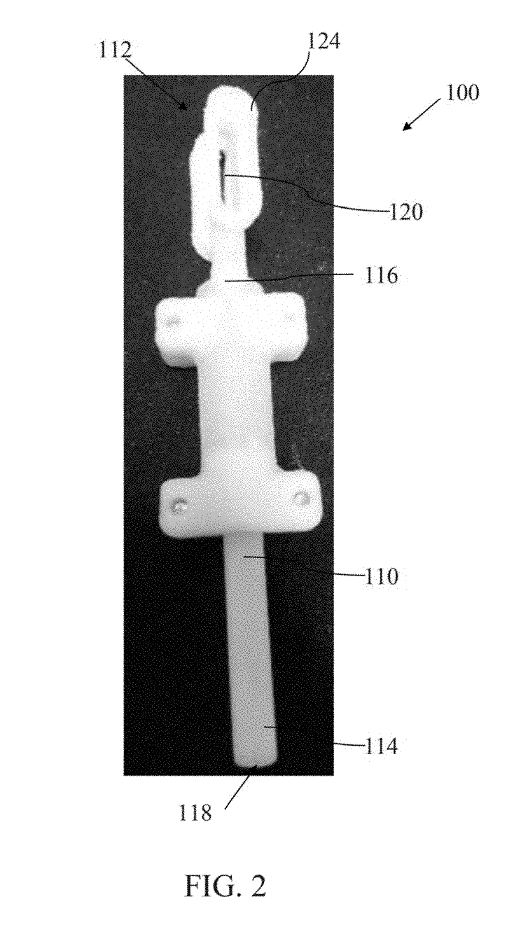

FIGS. 1 and 2 are perspective views of a pneumatic central airway stent removal device 100 according to an example. Central airway stent removal device 100 includes a shaft 110 and a pneumatic head 112. Shaft 110 includes a first pneumatic fitting 114 at a first end, a pneumatic head connection 116 at a second end, and a pneumatic aperture 118 running throughout shaft 110. In some examples, shaft 110 can be either rigid or flexible and configured for use with either rigid or flexible endoscopes. Further, shaft 110 is configured to convey negative or positive fluidic pressure from pneumatic fitting 114 to pneumatic head 112 via pneumatic aperture 118. Pneumatic fitting 114 is configured to couple to a surgical vacuum source such as a syringe or vacuum air line. In this example, negative pressure, e.g., vacuum, is conveyed through pneumatic aperture 118. Further, the vacuum supplied to pneumatic head 112 can be adjusted during the procedure to provide the user with the ability to increase or decrease the vacuum holding force between engagement ring 124 and the inner wall of the central airway stent.

Shaft 110 is coupleable to pneumatic head 112 at pneumatic head connection 116. Pneumatic head connection 116 is configured to provide fluidic engagement between shaft 110 and pneumatic head 112. In one example, shaft 110 and pneumatic head 112 are monolithic. In another example, pneumatic head 112 is selectively removable from shaft 110 at pneumatic head connection 116. In this example, various sizes of pneumatic heads 112 could be used with the same shaft 110, or vice-versa, in order to facilitate a variety of central airway stent diameters or different endoscopes. In this way, selectively removable pneumatic head 112 makes pneumatic central airway stent removal device 100 compatible with a variety of stent sizes, stent locations within the central airway, or patient size or anatomy, e.g., adult vs. child.

Pneumatic head 112 includes a manifold 120 and one or more bellows fittings 122. Each bellows fittings 122 include an engagement ring 124 and bellows walls 126. In some examples, manifold 120 of pneumatic head 112, visible in FIG. 2, consolidates fluid flow from bellows fittings 122 into pneumatic aperture 118. In an alternative positive pressure example of pneumatic central airway stent removal device 100, manifold 120 of pneumatic head 112 acts to separate and direct fluid flow from pneumatic aperture 118 to the one or more bellows fittings 122.

In some examples, engagement rings 124 of bellows fittings 122 are each configured to engage with an inner surface of a central airway stent to be removed, for example to engage in a manner that a fluidic seal is formed between each engagement ring 124 and portions of the inner surface of the central airway stent. To ensure engagement rings 124 contact the inner surface of a central airway stent when pneumatic head is being positioned within the central airway stent, bellows fittings 122 provide a radial mechanical spring force such that engagement rings 124 are biased to contact the inner surface of a central airway stent. In one example, bellows walls 126 have a selectively collapsible accordion structure and comprise a resilient material such as plastic, silicone rubber, other synthetic rubbers, or another suitable material. In this example, engagement ring 124 of bellows fittings 122 are configured to form a fluidic seal when engaged with central airway stents having an inner surface that is smooth and non-perforated, such as a synthetic coating or wall.

In an alternative, positive pressure example of pneumatic central airway stent removal device 100, engagement rings 124 of pneumatic head 112 are sealed such that positive pressure supplied to pneumatic head 112 causes engagement ring 124 to forcefully engage with the inner surface of the central airway stent. In other words, engagement ring 124 does not allow the inner surface of the central airway stent to be in fluidic engagement with the pressurized air. In this example, bellows fittings 122 do not collapse after engagement ring 124 engages with the inner surface of the central airway stent. Instead, sufficient engagement relies on the contact force on the inner surface of the central airway stent, and by extension, the friction force between engagement ring 124 and the inner surface of the central airway stent. The pressure needed to provide adequate friction force at the inner surface of the central airways stent can be in a range of 0 mmHg to 760 mmHg, for example 0 mmHg to 350 mmHg, or any pressure or range of pressures within the broader range. Further, the pressure supplied to pneumatic head 112 can be adjusted during the procedure to provide the user with the ability to increase or decrease the friction force between engagement ring 124 and the inner wall of the central airway stent. In another example of the positive pressure example of pneumatic central airway stent removal device 100, engagement ring 124 can further include hooks or other attaching devices, such that when positive pressure is applied to bellows fittings 122, the hooks of engagement ring 124 couple to the inner surface or distal end of the central airway stent. In this example, the hooks aid in or fully provide sufficient engagement between engagement ring 124 and the inner surface of the central airway stent for central airway stent removal.

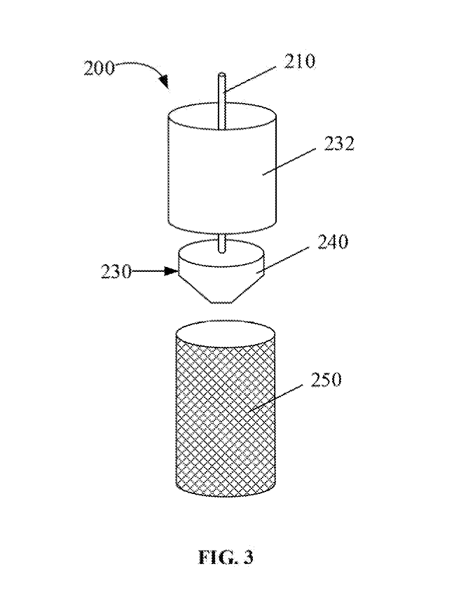

FIG. 3 is a perspective view of a mechanical central airway stent removal device 200 according to an example, shown with a central airway stent 250. In some examples, mechanical central airway stent removal device 200 includes a shaft 210, a collet 230 coupled to a distal end of shaft 210, and a separator 232 coaxially coupled to shaft 210. Collet 230 further includes engagement walls 240. Separator 232 is configured to slide coaxially along the length of shaft 210 and extending distally beyond collet 230.

In some examples, shaft 110 can be either rigid or flexible and configured for use with either rigid or flexible endoscopes. Further, separator 232 can be configured for use with either rigid or flexible endoscopes.

In some examples, engagement walls 240 of collet 230 are configured to engage with the inner surface of central airway stent 250. In one example, collet 230 can be configured such that engagement walls 240 resiliently deform when entering central airway stent 250. But, when collet 230 begins motion in the opposite direction, i.e., pulling collet 230 back out of central airway stent 250, engagement walls 240 radially expand and frictionally engage with inner surface of central airway stent 250. This frictional engagement between engagement walls 240 and the inner surface of central airway stent 250 is such that removal of collet 230 also acts to remove central airway stent 250 from central airway 260.

In other examples, engagement walls 240 can be forcefully expanded using an internal wedge, balloon, or other suitable spreading mechanism. In yet another example, engagement walls 240 can be configured to closely match the inner diameter of central airway stent 250, and with separator 232 positioned exterior to central airway stent 250, central airway stent 250 can be captured between engagement walls 240 and separator 232 for placement, repositioning, or removal.

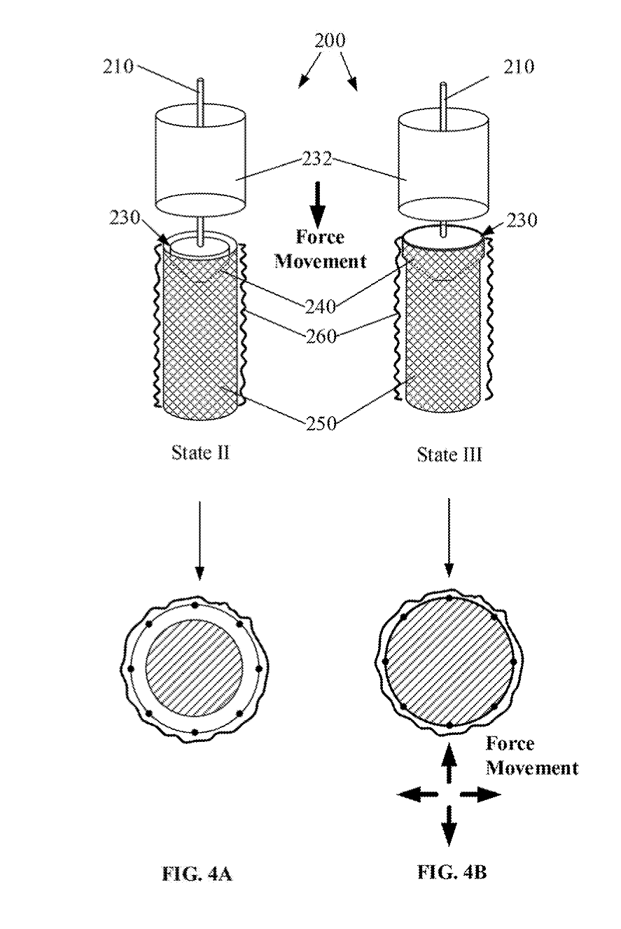

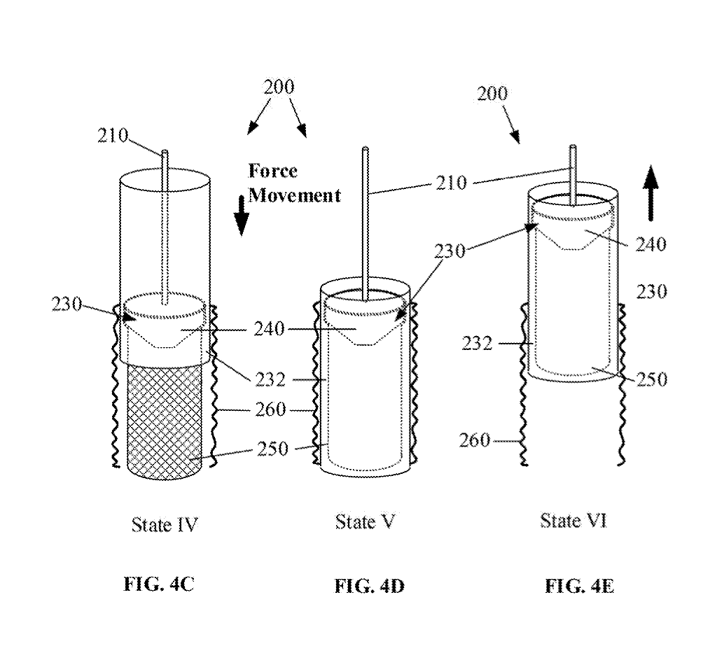

In some examples of mechanical central airway stent removal device 200, separator 232 may be configured to coaxially slide around the exterior of central airway stent 250, between an outer wall of the stent and the wall of central airway 260. In this example, separator 232 can sever any tissue engagement between central airway stent 250 and the wall of central airway 260, thereby aiding in removal of central airway stent 250. In one example, separator 232 can act in concert with collet 230, as is depicted in FIGS. 3 and 4A-4E. In another example, separator 232 can act independently to separate central airway stent 250 from the wall of central airway 260 and then to remove central airway stent 250 once the stent is captured by separator 232. Further, separator 232 can be used in concert with any other example as described herein.

FIGS. 4A-4E depict a sequential removal process in which mechanical central airway stent removal device 200 is used to remove central airway stent 250 from a central airway. FIG. 4A depicts collet 230 entering the inner lumen central airway stent 250. FIG. 4B depicts collet 230 engaging with the inner surface of central airway stent 250. Here, engagement walls 240 are mechanically engaged with the inner surface of central airway stent 250. At this point friction forces between engagement walls 240 and the inner surface of central airway stent 250 are such that movement of central airway stent 250 relative to collet 230 is restricted. FIG. 4C depicts separator 240 sliding over the exterior wall of central airway stent 250 but within central airway 260 itself. This action severs any connection (e.g. connective tissue, suture, barb, etc.) between the exterior wall of central airway stent 250 and the inner tissue of central airway 260. One aspect of this movement is that the engagement between collet 230 and the inner surface of central airway stent 250 restricts central airway stent 250 from sliding distally in response to the engaging forces of separator 240. FIG. 4D depicts separator 240 having mostly or fully captured central airway stent 250. FIG. 4E depicts the removal of central airway stent 250 after the stent has been captured coaxially between collet 230 and separator 240.

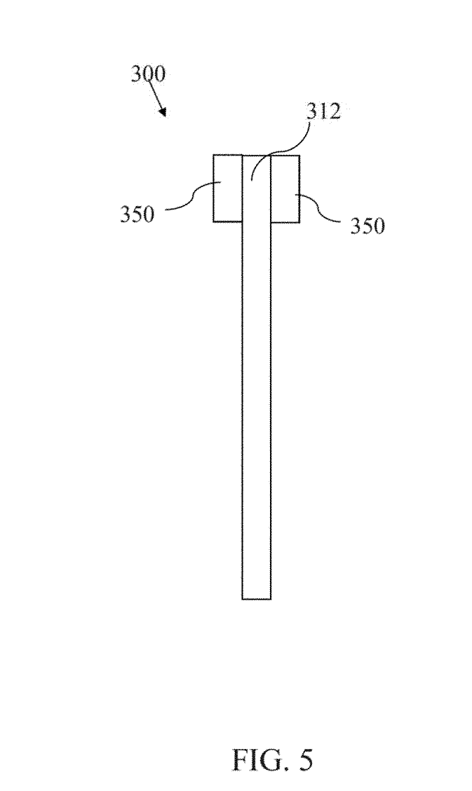

In another example, as depicted in FIG. 5, a magnetic central airway stent removal device 300 can be used for central airway stent removal. In this example a magnetic head 312 can replace either pneumatic head 112 of pneumatic central airway stent removal device 100 or collet 230 of mechanical central airway stent removal device 200. In this example magnetic head 312 includes one or more magnets 350. Two magnets are depicted in FIG. 5, but in other examples one magnet (e.g., a disc- or donut-shaped magnet coupled to or on magnetic head 312) or more than two magnets (e.g., four magnets arranged relative to another at sequential 90-degree positions around magnetic head 312) can be used. Magnets 350 can comprise ceramic, neodymium, samarium cobalt, or other suitable magnetic material.

Magnets 350 are configured to engage with a central airway stent having ferrous or other magnetic properties. In one example, magnetic head 312 is configured to enter the interior portion of the central airway stent and magnetically engage with a material of the central airway stent. In one example, magnets 350 can be sized and otherwise configured such that the central airway stent collapses onto magnetic head 312 because of magnetic force therebetween or is held in place for a tool or separator, such as separator 240, to be used with magnetic central airway stent removal device 300 to disengage the central airway stent from the central airway before magnetic central airway stent removal device 300, with the stent magnetically coupled thereto, is retracted (in the case of stent removal) or advanced (in the case of either stent placement or repositioning).

In another example, magnets 350 can be selectively electro-magnetic. In other words, magnet 350 can include a ferrous core with a coil of wire surrounding the core which is electrically coupled to an external electrical power source. In this example, magnets 350 can be selectively magnetized by supplying or not supplying electrical power to magnets 350. In this example, magnetic central airway stent removal device 300 can be used to position magnets 350 within the central airway stent in a non-magnetized state, then when properly positioned, magnets 350 can be magnetized via power supply to engage with the inner surface of the central airway stent.

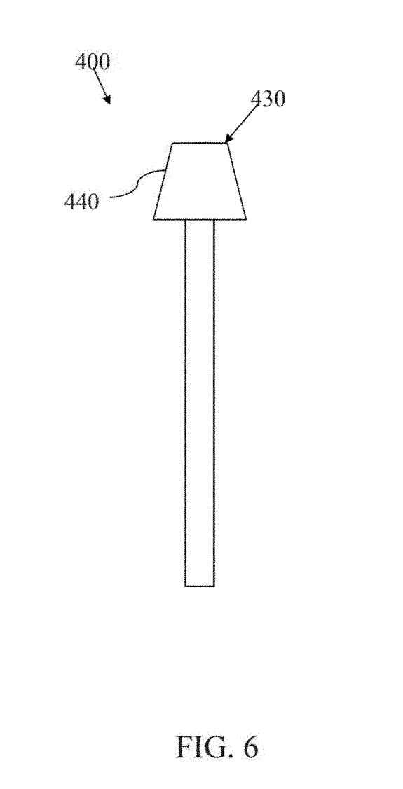

In another example, as depicted in FIG. 6, a thermal central airway stent removal device 400 uses thermally activated shape memory alloys or other materials to establish frictional engagement with a central airway stent to position, reposition or remove the stent. In this example, thermal collet 430 and engagement walls 440 can replace collet 230 and engagement walls 240, respectively, of mechanical central airway stent removal device 200 as previously described. The particular shape and configuration of thermal collet 430 and engagement walls 440 can vary from those depicted in FIG. 6 in other examples.

In this example, engagement walls 440 can expand when subject to elevated temperature using a shape memory alloy, such as nickel titanium, or other suitable shape memory alloy. At room temperature, engagement walls 440 are in an unexpanded state, then, when subject to thermal energy such as body heat from the patient or an external source, engagement walls 440 can radially expand to engage with the inner surface of the central airway stent. In an expanded state, engagement walls 440 can be configured such that the engagement between engagement walls 440 and the inner surface of the central airway stent is subject to friction forces that are suitable for positioning or removal of the inner surface of the central airway stent.

In an alternative example, thermal energy can be created by subjecting thermal collet 430 to electrical energy. In this example thermal collet 430 is configured to be a resistive heat generating element of an electrical circuit. Thus, when electrical energy is applied to thermal collet 430, the shape memory alloy of engagement walls 440 begins to heat and therefore expand.

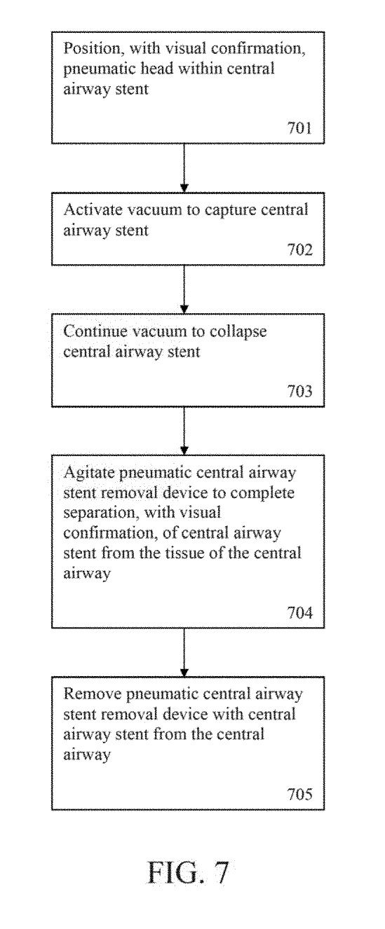

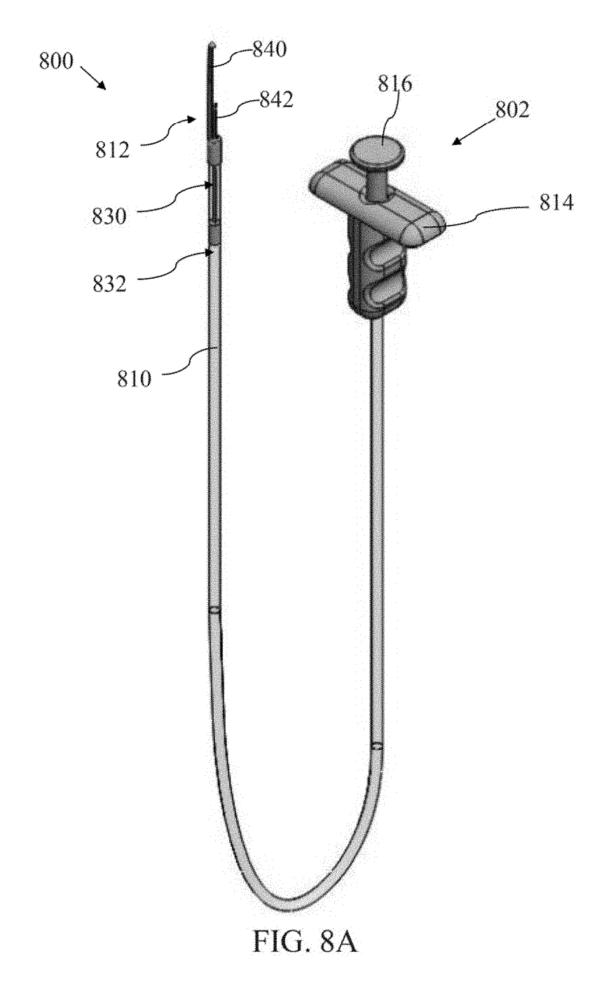

FIG. 7 is a flowchart of a method of use of pneumatic central airway stent removal device 100 for removing a central airway stent with the aid of an endoscope. Though FIG. 7 relates to pneumatic central airway stent removal device 100, one of ordinary skill in the art will appreciate that the method is similar for examples of mechanical central airway stent removal device 200, magnetic central airway stent removal device 300, and thermal central airway stent removal device 400.

In step (701), and under direct visual observation and confirmation using an endoscope (which also can be performed or maintained at any point in the method of FIG. 7), a user (such as a clinician) advances pneumatic central airway stent removal device 100 to a stent in a patient's central airway and positions pneumatic head 112 within the central airway stent. The user can visually ensure that engagement rings 124 are positioned such that sealable engagement with the inner surface of the central airway stent can occur.

In step (702), the user activates a vacuum to apply negative pressure to the central airway stent by pneumatic head 112 of pneumatic central airway stent removal device 100, thereby causing engagement rings 124 to become sealably engaged with the inner surface of the central airway stent to capture central airway stent.

In step (703), the user can continue vacuum pressure, and if necessary increase or decrease the amount of pressure, to cause the central airway stent to collapse. The vacuum needed to provide adequate holding force at the inner surface of the central airways stent such that collapse can occur can be in a range of 0 mmHg to 760 mmHg. The collapsibility of bellows fittings 122 causes the central airway stent to collapse and thereby retract from the tissue of the central airway.

In step (704), the user can agitate pneumatic central airway stent removal device 100, now pneumatically coupled with the central airway stent, to complete separation of the central airway stent from the tissue of the central airway.

In step (705), pneumatic central airway stent removal device 100 can be retracted and removed from the central airway and along with it, the collapsed and detached central airway stent. In other examples in which the central airway stent is being repositioned instead of removed, pneumatic central airway stent removal device 100 can be advanced or retracted until the central airway stent is positioned as desired within an airway, and the pneumatic pressure can be released, for example gradually, to cause the central airway stent to expand and be released from pneumatic central airway stent removal device 100. The final position of the central airway stent can be confirmed by direct visual observation by the clinician using the endoscope before the pneumatic central airway stent removal device 100 is then retracted and removed, leaving behind the central airway stent.

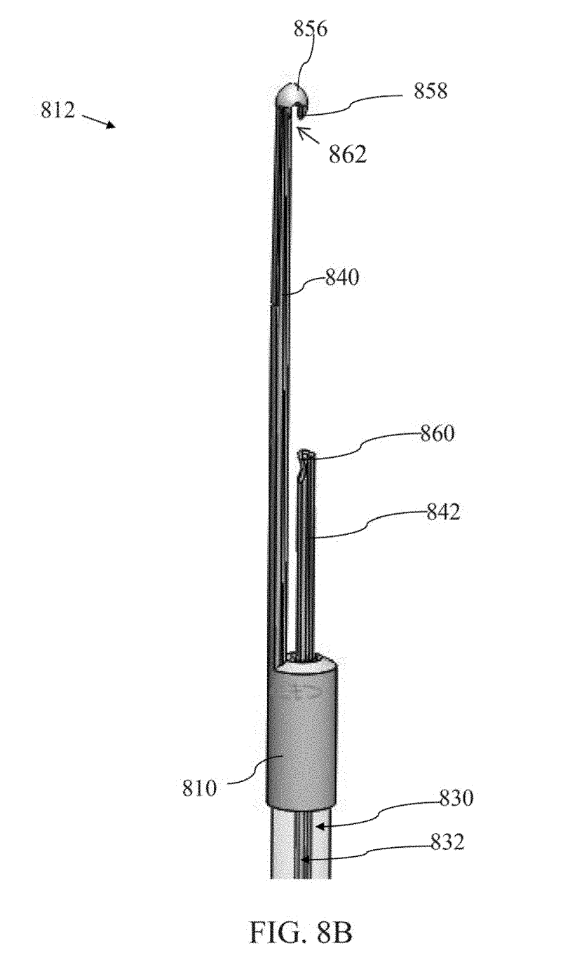

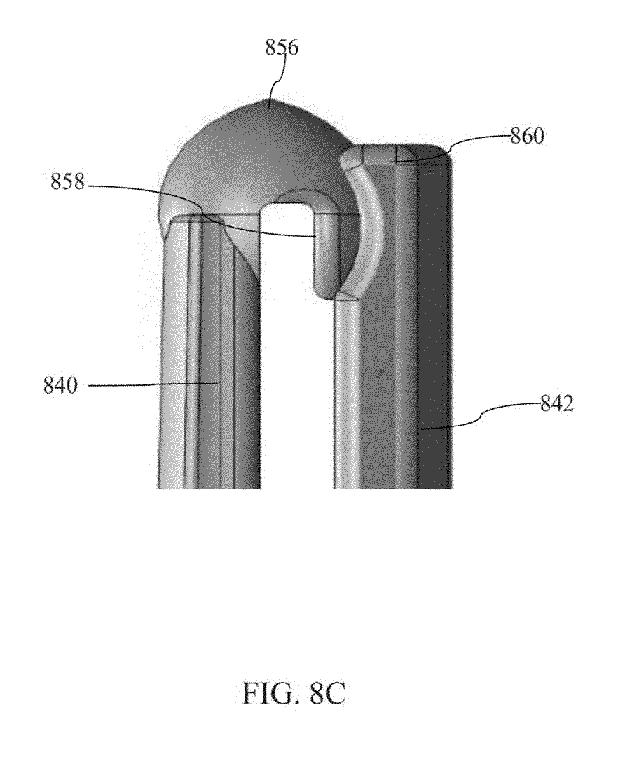

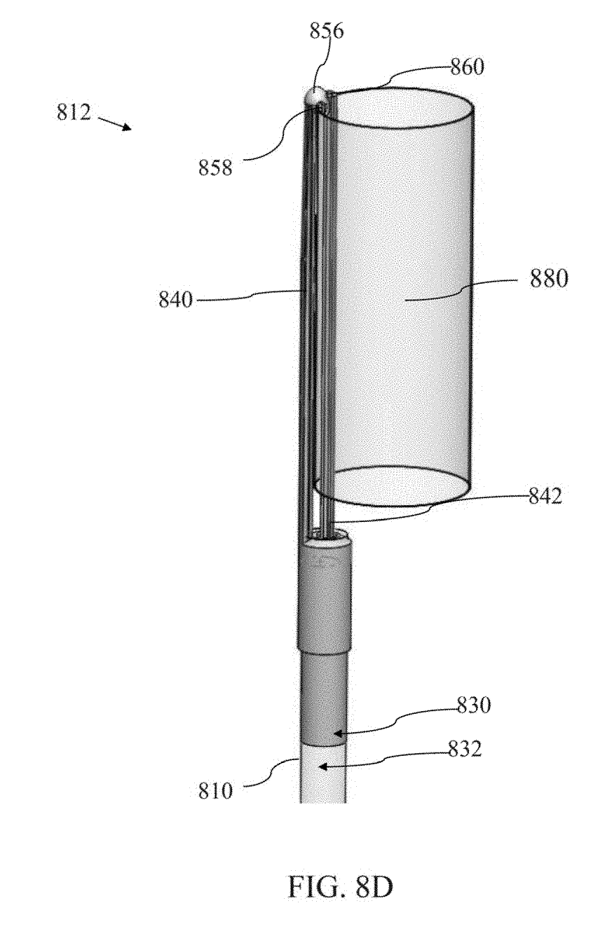

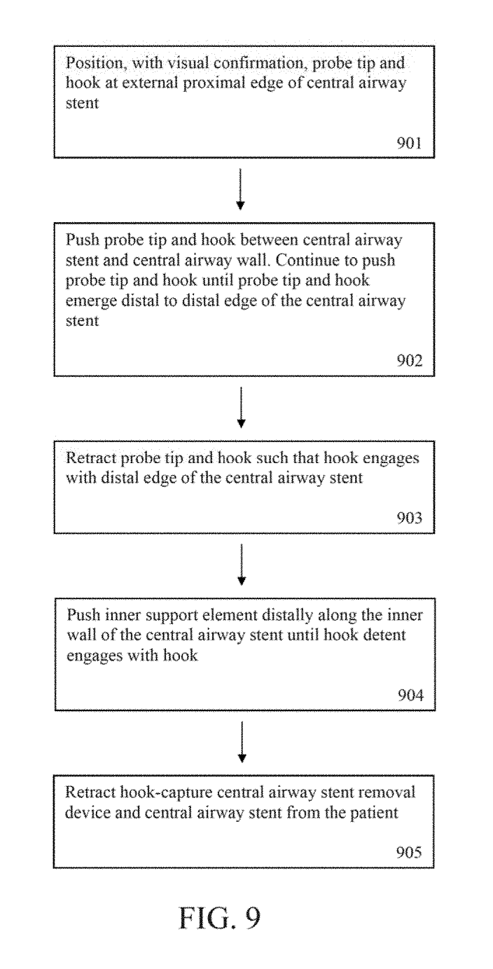

FIGS. 8A-8D illustrate another example central airway stent removal device 800 that includes a mechanical, hook-capture removal mechanism. Referring to FIG. 8A, hook-capture central airway stent removal device 800 may include a hub assembly 802, a elongated shaft 810, and a hook mechanism 812. Central airway stent removal device 800 that can be used with rigid or flexible bronchoscopy for positioning, repositioning, or removal of central airway stents. For example, elongated shaft 810 may be flexible and configured to be introduced through a working channel (e.g., inner lumen) of a flexible endoscope as described further below with respect to FIG. 9. In other examples, elongated shaft 810 may be ridged.

In some examples, elongated shaft 810 is coupled to hub assembly 802 at a proximal end and hook mechanism 812 at a distal end of the device. Hub assembly 802 is used to operate hook mechanism 812 and may include a handle 814 and a controller 816 arranged on handle 814. In examples controller 816 is arranged on handle 814 such that, with a single hand, controller 816 can be physically manipulated by a user while the user grips handle 814 to toggle the mechanical engagement of hook mechanism 812.

In some examples, elongated shaft 810 can include an inner lumen 830 arranged coaxially along the length of elongated shaft 810. Elongated shaft 810 further includes a control wire 832 located within inner lumen 830 and operably coupled (at a proximal end of control wire 832) with hub assembly 802, specifically controller 816.

Hook mechanism 812 is coupled to a distal end of elongated shaft 810 and includes a receiving platform 840 and a hook element 858, and an inner support element 842 coupled to a distal end of control wire 832. In some examples, inner support element 842 can comprise part of control wire 832. Control wire 832 and, in some examples, inner support element 842 can further be configured to selectively and slidably move within inner lumen 830 of elongated shaft 810 via user actuation of controller 816. Referring also to FIG. 8B, receiving platform 840 is offset from inner lumen 830 and inner support element 842 such that inner support element 842 can slidably move adjacent to receiving platform 840.

Hook mechanism 812 also includes a probe tip 856, with both probe tip 856 and hook element 858 arranged at a distal end of receiving platform 840. Hook element 858 defines a proximal opening slot 862 for receiving a portion of a central airway stent 880. Inner support element 842 can include, at a distal end thereof, a hook detent 860 suitable for engaging with the exterior of hook element 858 (discussed in more detail below with respect to FIG. 8C). In some examples, probe tip 856 can be configured to have a pointed tip and smooth transitional walls suitable for entering the space between the external wall of central airway stent 880 and the central airway wall. In other examples, probe tip 856 can have an elongated point or other configuration that provides increased stent-wall separation ability. Hook element 858 can be configured to selectively engage with a distal edge of central airway stent 880. For example, the spacing with hook element 858 (e.g., slot 862) or another characteristic of the size, shape or general configuration of hook element 858 can be configured to fit over, grasp, pull or otherwise engage with the wall of central airway stent 880 in use. In other examples not depicted, inner support element 842 can be offset from receiving platform 840 and include another hook feature, such as one similar to hook element 858, such that both inner support element 842 and hook element 830 can engage with a different (e.g., adjacent) portion of the distal edge of central airway stent 880.

Referring also to FIGS. 8C and 8D, inner support element 842 can move distally and adjacent to receiving platform 840 until hook detent 860 engages with hook element 858. Once hook detent 860 and hook element 858 are engaged, an enclosure is formed between receiving platform 840 and inner support element 842, in which the wall of central airway stent 880 can be captured in use.

In some examples, controller 816 can include a spring such that controller 816 is biased with inner support element 842 retracted. In an alternative example, controller 816 can include a spring such that controller 816 is biased in a position with inner support element 842 being fully advanced. In these or other examples, controller 816 also can include a locking element to enable a user to selectively lock controller 816 in a depressed or released position, or any position therebetween.

FIG. 9 is a flowchart of a method of use of hook-capture central airway stent removal device 800 for removing central airway stent 880 with the aid of an endoscope.

In step (901), and under direct visual observation and confirmation using an endoscope (which also can be performed or maintained at any point in the method of FIG. 9), a user (such as a clinician) advances central airway stent removal device 800 to stent 880 in a patient's central airway and positions, with visual confirmation, probe tip 856, hook element 858, and receiving platform 840 of hook mechanism 812 at an external proximal edge of central airway stent at a target treatment site within the body of a patient.

Hook mechanism 812 may be navigated to the target treatment site using any suitable technique. For example, a flexible endoscope that defines a working channel (e.g., inner lumen) may be navigated to the target treatment site including the central airway stent using a suitable technique. Hook mechanism 812 may then be introduced into the working channel of the endoscope and elongated shaft 810 may be forced through the working channel to position hook mechanism 812 at the distal end of the endoscope. In such example, the flexibility of elongated shaft 810 may permit the device 800 the ability to bend and shape to the contours of the flexible endoscope allowing hook mechanism 812 to be navigated to the target treatment site.

In step (902), the clinician, via hub assembly 802, pushes probe tip 856 and hook element 858 between the exterior wall of the central airway stent and the central airway wall along the exterior surface of the central airway stent 880, until probe tip 856 and hook element 858 emerge from between the exterior wall of the central airway stent and the central airway wall at the distal edge of the central airway stent. In this configuration (e.g., FIG. 8D), the receiving platform 840 will be positioned between the central airway wall and the exterior surface of stent 880, which may help to separate any connective tissue between stent 880 and the central airway.

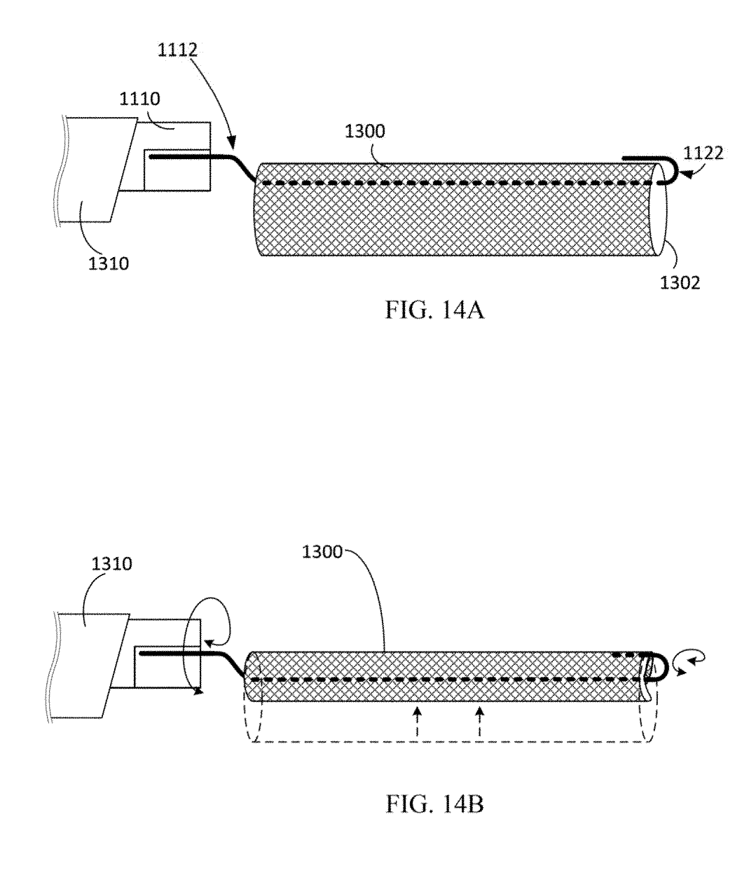

In other example, hook element 858 and receiving platform 840 may be initially passed through the inner lumen of stent 880 rather than between the central airway wall and stent 880 (e.g., similar to the configuration shown in FIG. 14A). In such configurations, inner support element 842 (if present) may be passed between the outer surface of stent 880 and the central airway wall.

In step (903), the user can retract probe tip 856 and hook element 858, via handle 814, such that hook element 858 engages with the distal edge of central airway stent 880. With visual guidance from the endoscope, the user can confirm engagement when hook element 858 visibly cups the distal edge of central airway stent 880. Rotation of probe tip 856 and hook element 858, or selective readvancement or retraction, may be necessary to engage hook element 858 with a distal edge of the central airway stent.

In step (904), the user can manipulate controller 816 to advance inner support element 842, via control wire 832, distally along the inner wall (or exterior wall depending on the manner of hook element 858 engagement) of the central airway stent until hook detent 860 engages with hook element 858. Visual confirmation can be used to ensure inner support element 842 moves within the central airway stent along the inner wall of the central airway stent, as opposed to outside the central airway stent along the exterior wall of the central airway stent.

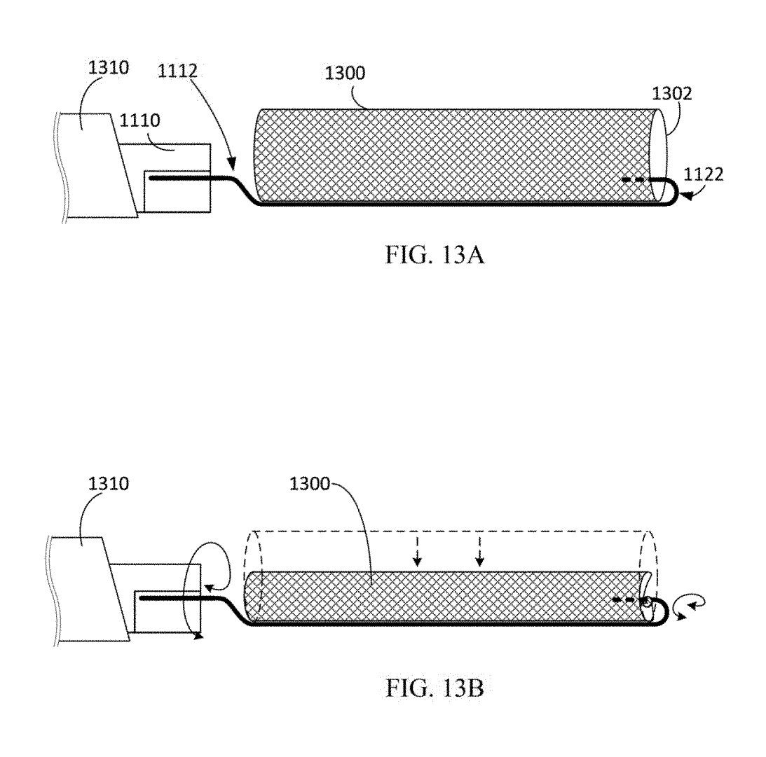

In step (905), the user can retract hook-capture central airway stent removal device 800 and, along with it, the central airway stent from the patient. If the central airway stent is difficult to remove because of tissue engagement with the central airway, hook-capture central airway stent removal device 800 can be manipulated such that receiving platform 840 and inner support element 842 can agitate, slide between, twist, or otherwise manipulate the wall of the central airway stent such that the tissue engagement is broken. Additionally, or alternatively, to help facilitate removal of stent 880, hook mechanism 812 may be rotated about the longitudinal axis of elongated shaft 810 relative to stent 880. The relative rotation, as described further below with respect to FIGS. 13A to 14B, may be used to cause stent 880 to rollup and collapse in on itself, making the stent easier to be removed. During removal, stent 880 and hook mechanism 812 may be withdrawn into the working channel of the flexible endoscope and both the endoscope and stent removal device 800 may be removed from the patient.

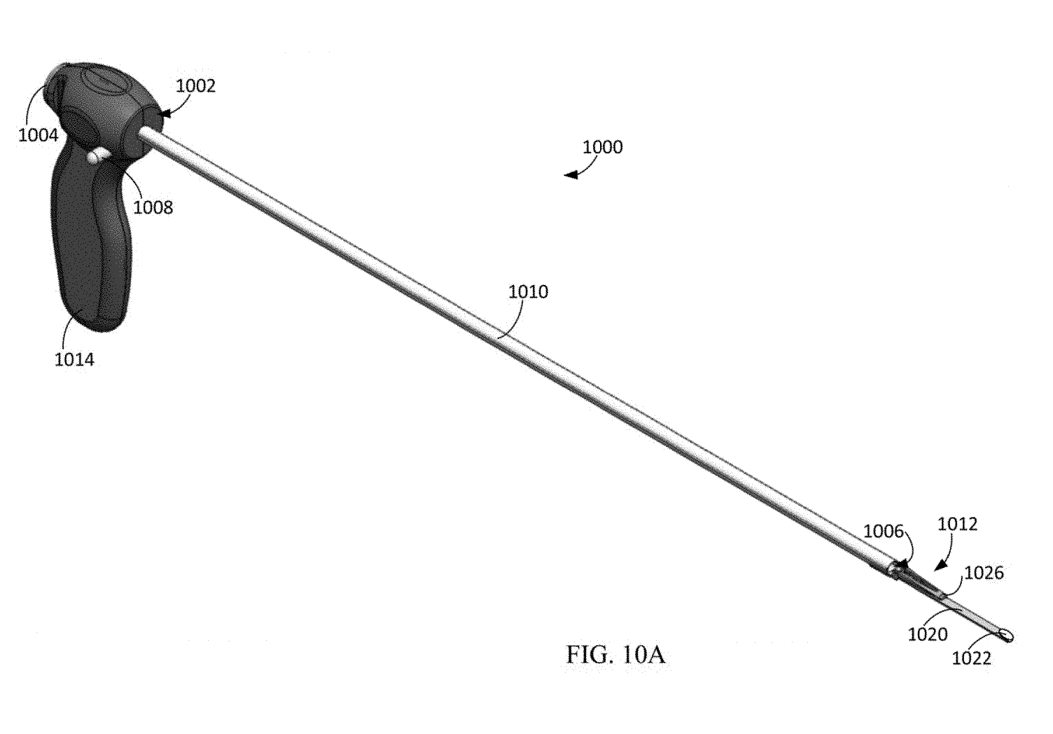

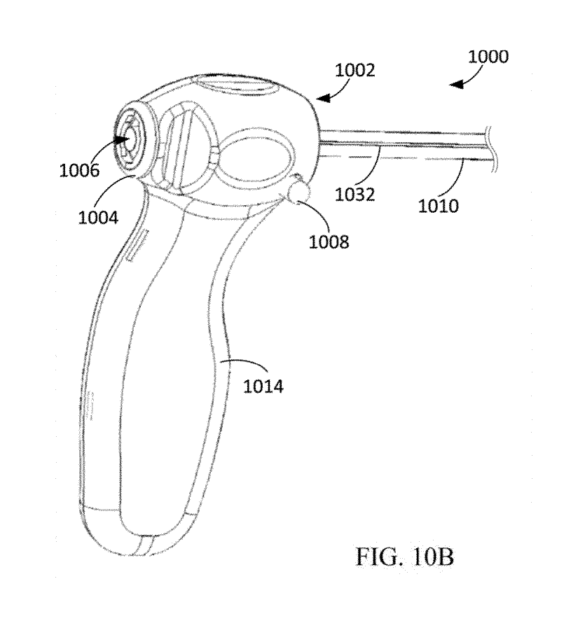

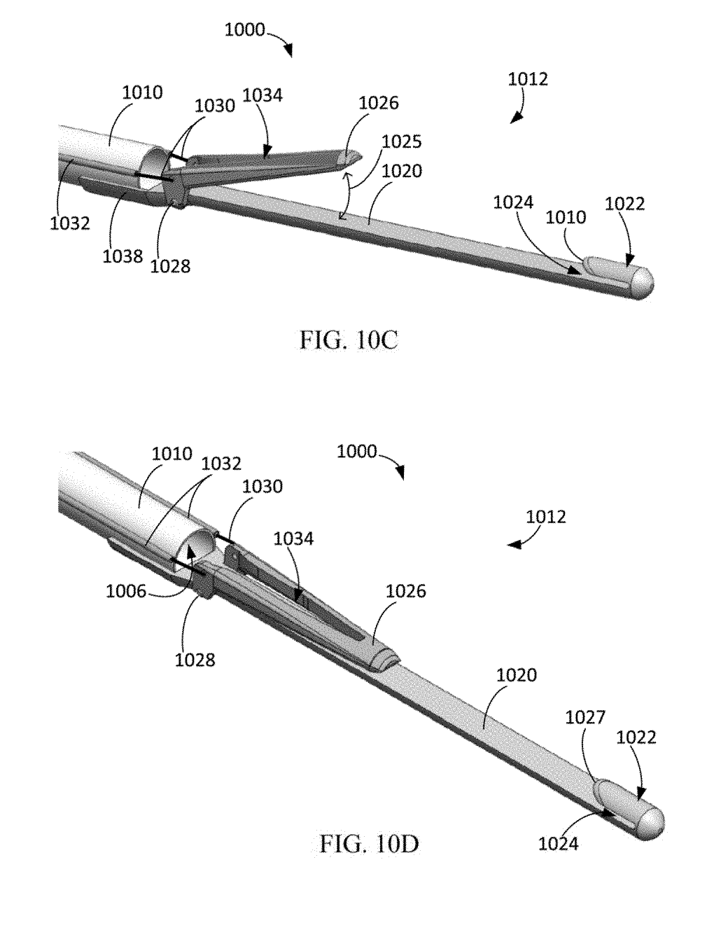

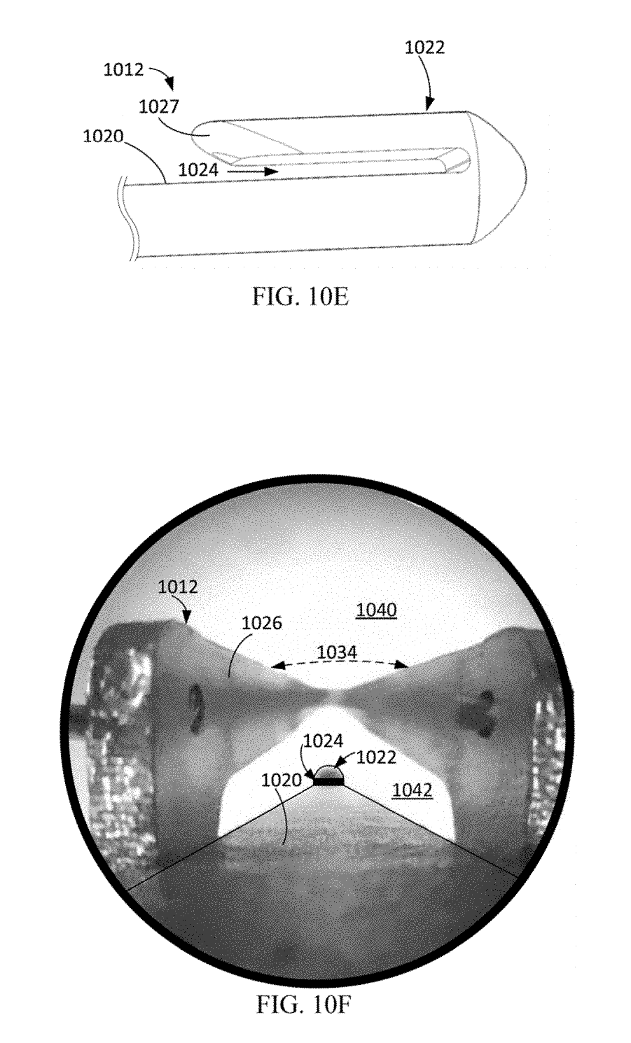

In other examples in which the central airway stent is being repositioned instead of removed, hook-capture central airway stent removal device 800 can be advanced or retracted until the central airway stent is positioned as desired within an airway, and inner support element 842 can be retracted, hook element 858 can be released from the distal edge of the central airway stent, and the receiving platform 840 can be retracted along the exterior wall of the central airway stent. The final position of the central airway stent can be confirmed by direct visual observation by the clinician using the endoscope before the hook-capture central airway stent removal device 800 is then retracted and removed, leaving behind the central airway stent. FIGS. 10A-10F illustrate various views of another example central airway stent removal device 1000 that includes a mechanical, hook-capture removal mechanism. Central airway stent removal device 1000 includes an elongated shaft 1010, a hub assembly 1002 coupled to a proximal end of shaft 1010, and a hook mechanism 1012 coupled to a distal end of shaft 1010.

Shaft 1010 may by rigid or flexible and configured for use with either rigid of flexible endoscopes. In some examples, hub assembly 1002 and shaft 1010 may define a central lumen 1006 configured to receive an endoscope. Endoscopes (including telescopes such as, for example, 10320 AA Hopkins® Straight Forward Telescope, diameter 5.5 mm, length 50 cm) are conventionally known and will be well understood by those in the art. The inner diameter of the central lumen 1006 may be sized depending on the type of endoscope intended to be received. Conventional endoscope diameters may range from about 3.5 mm to about 8.5 mm. Using the endoscope in conjunction with central airway stent removal device 1000 allows the clinician to visually see the engagement of hook mechanism 1012 with the central airway stent. In some examples, central lumen 1006 may extend from the proximal end of hub assembly 1002 through the entire length of shaft 1010. In some example, shaft 1010 may be constructed as a rigid tube (e.g., metal tube) and configured to receive a rigid endoscope through the inner lumen 1006 of the rigid tube.

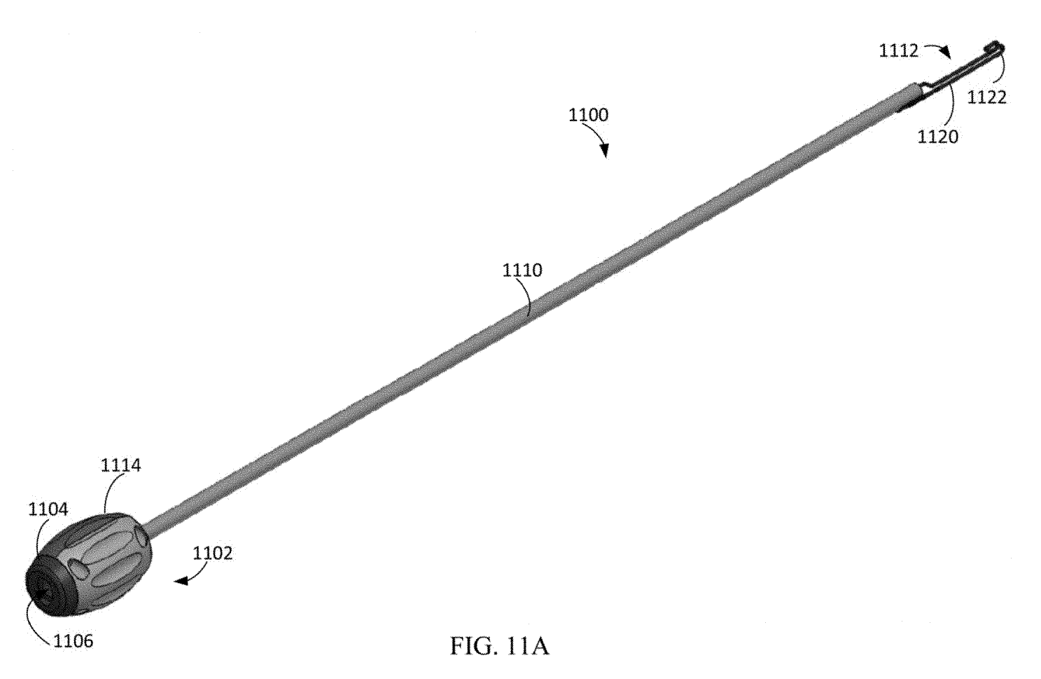

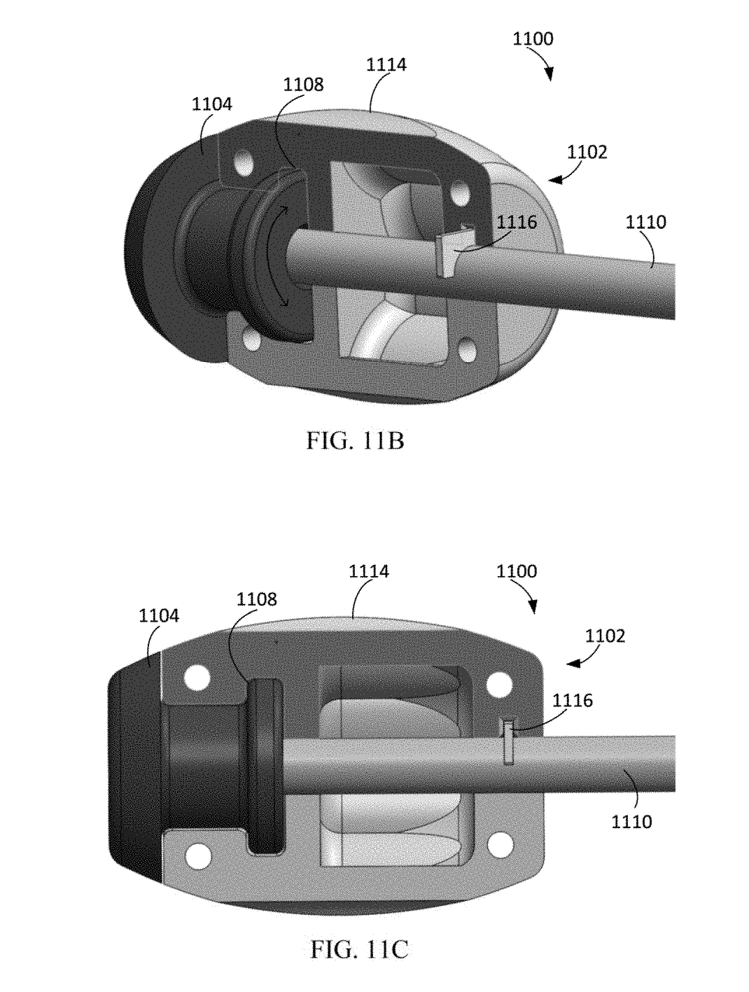

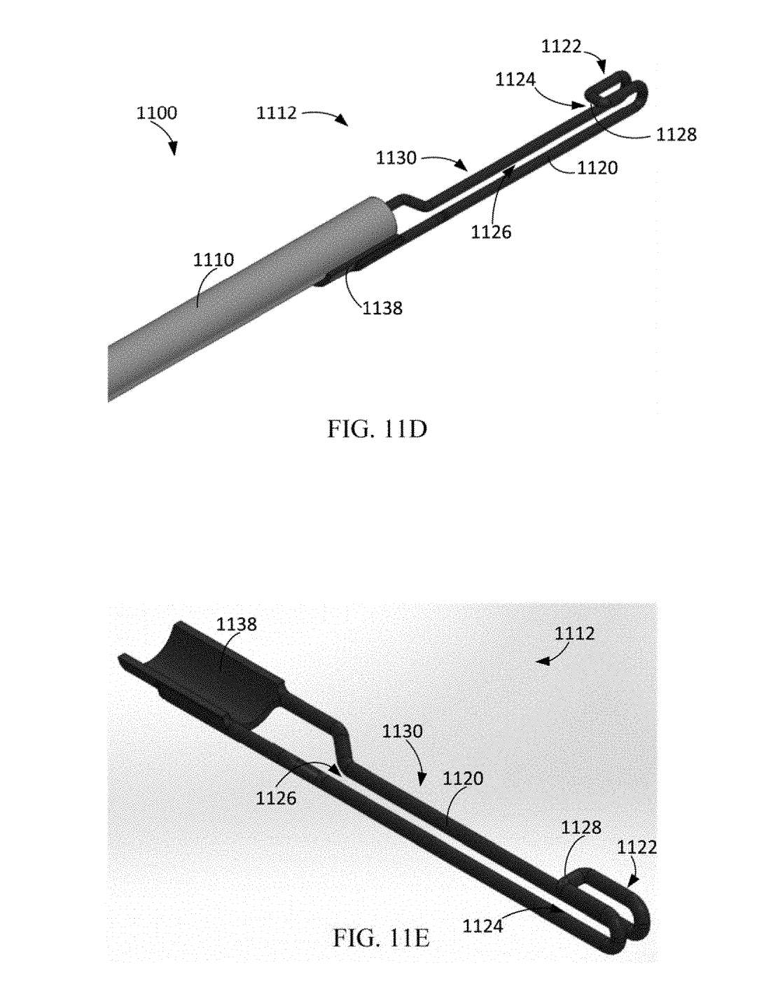

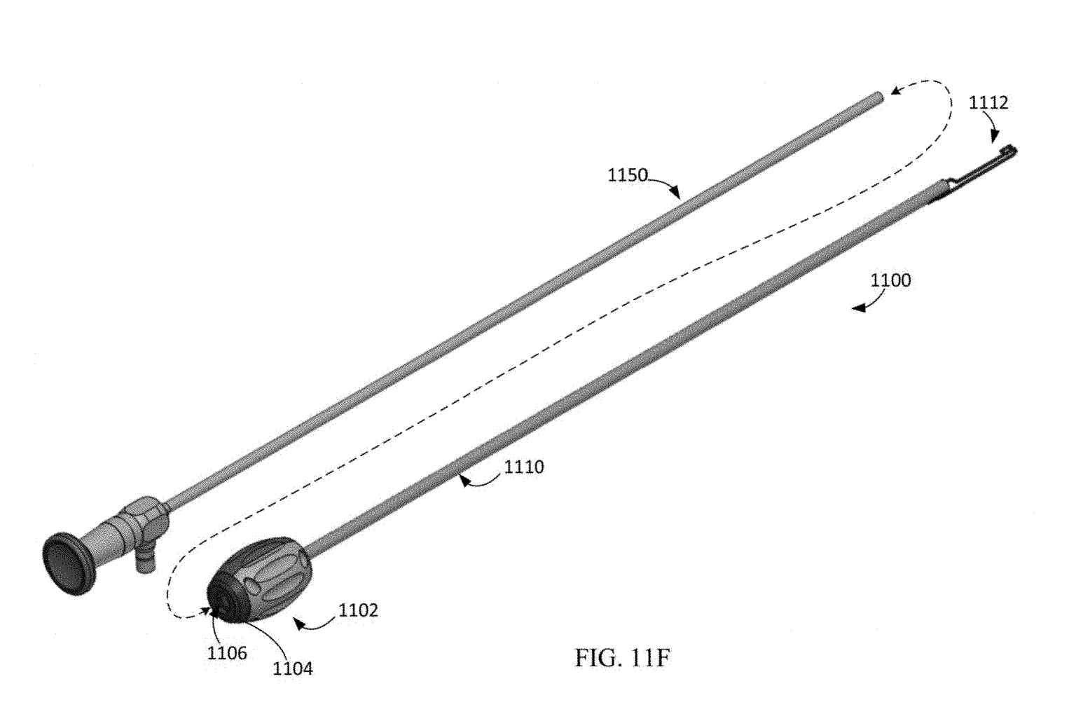

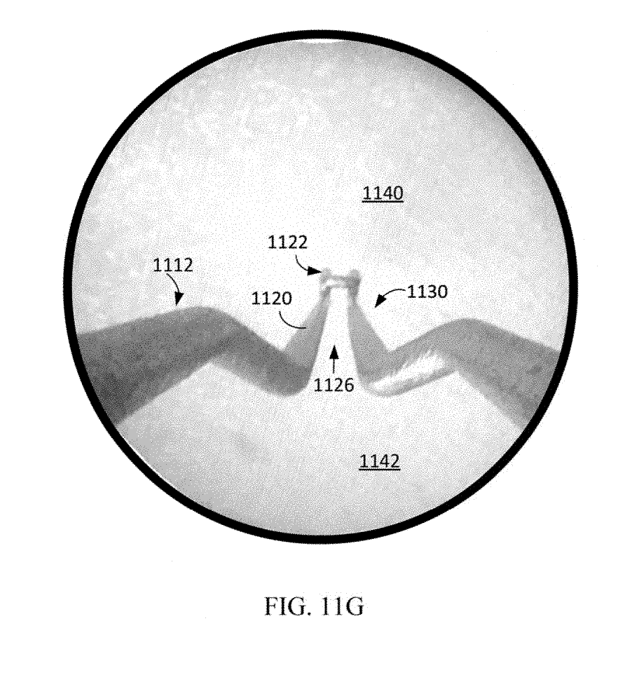

Hub assembly 1002 may include a grip 1014 that can be grasped by a clinician and used to help navigate hook mechanism 1012 to a target site and manipulate hook mechanism 1012 to engage or disengage with a central airway stent as needed. Grip 1014 may take on any suitable design. In some examples, grip 1014 may include a pistol grip as shown in FIGS. 10A and 10B to allow the clinician to easily grab hub assembly 1002 and provide torque and control over shaft 1010. In other examples, as describe further below with respect to FIGS. 11A-11C, grip 1014 may include a barrel grip aligned coaxially and/or concentrically with shaft 1010. The barrel grip may allow for convenient rotation of shaft 1010 in order to help remove the central airway stent from its deployment site as well as providing a central position for the clinician to grip and manipulate the device. In some examples, grip 1014 may be fixedly connected relative to shaft 1010 such that grip 1014 and shaft 1010 move as a single unit.

The proximal end of hub assembly 1002 also includes an adaptor 1006 configured to mechanically interlock (e.g., twist lock) with a connector of an endoscope once the endoscope is received within central lumen 1006. In some examples, the adaptor 1006 may be fixedly connected relative to shaft 1010 and grip 1014 and in other examples adaptor 1006 may be rotatable relative to shaft 1010 and/or grip 1014. Having adaptor 1006 be rotatable relative to shaft 1010 and grip 1014 may allow for the shaft 1010 and hook mechanism 1012 to rotate independent of the endoscope during the procedure without causing the endoscope to rotate or become disengaged from central airway stent removal device 1000. Shaft 1010 may be constructed using a ridged or flexible materials. Suitable rigid materials include, but are not limited to, surgical grade metal such as stainless steel. Flexible materials may include, but are not limited to, reinforced polymeric tubes (e.g., coil reinforced PEBAX).

As shown in FIG. 10B, hook mechanism 1012 includes an elongated receiving platform 1020 that extends distally away from the distal end of shaft 1010 along the longitudinal axis of shaft 1010 and a hook element 1022 at the distal end of receiving platform 1020 that curls back toward hub assembly 1002. In this way, hook element 1022 defines a receiving slot 1024 that opens towards the proximal end of the device 1000 and is closed at the distal end of the device. Receiving slot 1024 is configured to receive a portion of the sidewall of a central airway stent with the remaining sidewall of the stent received against receiving platform 1020. Once received in receiving slot 1024, the central airway stent if prevented from advancing distally due to hook element 1022.

In some examples, the depth of slot 1024 may be relatively small compared to the overall length of the stent and receiving platform 1024. For example, the depth of slot 1024 (e.g., length measured in the direction of the longitudinal axis of shaft 1010) may be about 0.5 cm to about 2 cm. The slot height may be about 0.1 mm to about 2 mm but other heights are also envisioned depending on the relative thickness of the sidewall of the central airway stent. In some examples, the entrance for slot 1024 may include a tapered section 1027 such that the entrance of slot 1024 can help guide or funnel an end of the central airway stent into slot 1024 during the stent retrieval process.

In some examples, the total length receiving platform 1020 added with the depth of slot 1024 may be sized to accommodate the total length of central airway stents. Standardize lengths for central airway stents include 40 mm, 80 mm, and 120 mm. Thus, in some examples, the total length of receiving platform 1020 and slot 1024 combined may be equal to or greater than about 40 mm, equal to or greater than about 80 mm, or equal to or greater than about 120 mm.

In some examples, hook mechanism 1012 may be radially offset from the longitudinal axis of shaft 1010 with hook element 1022 hooking towards the longitudinal axis rather than away from longitudinal axis. This configuration may help prevent hook element 1022 or hook mechanism 1012 from unintentionally catching or contacting on the, bronchoscopy guide tube (e.g., guide tube 1310 in FIG. 13A), the central airway stent, or bodily tissue of the patient during use of the device. Additionally, or alternatively, the radially outer surface of receiving platform 1020 and hook element 1022 as well as the distal tip of hook element 1022 may be rounded or otherwise blunted to help reduce and prevent trauma between hook mechanism 1012 and the tissue of the patient at the treatment site. Additionally, the cross-sectional diameter of shaft 1010 and hook mechanism 1012 may be sized to fit within a bronchoscopy guide tube (e.g., guide tube 1310 in FIG. 13A).

In some examples, hook mechanism 1012 also includes optional retaining arm 1026 configured to operably engage with the central airway stent to help secure the stent against receiving platform 1020 during retrieval. For example, optional retaining arm 1026 may be pivotally connected to receiving platform 1020 so that it opens and closes towards the proximal end of receiving platform 1020 about pivot point 1028 (shown by arrows 1025 in FIG. 10C). Using a suitable control mechanism, retaining arm 1026 may be transitioned from an open configuration to a closed configuration similar to the action mechanism of a pair of forceps. As shown in FIGS. 10A and 10B, retaining arm 1026 may be toggled using one or more control wires 1030 connected to retaining arm 1026 at a position offset to pivot point 1028, with the opposite ends of control wires 1030 being received by hub assembly 1002 and actuated by control lever 1008. In some examples, control wires 1030 may be received within a respective a control tubes 1032 attached to shaft 1010 that houses and helps protect the respective wires 1030 from the surrounding environment.

Optional retaining arm 1026 may be a u-shaped arm having the ends of the u-shape pivotably attached to radially opposite sides of receiving platform 1020. The distal end of retaining arm 1026 (e.g., the crest of the u-shape) may extend towards hook element 1022 and aligned to operate in conjunction with slot 1024 to receive and secure the central airway stent against receiving platform 1020. The u-shape construction of retaining arm 1026 defines a slot 1034 that helps increase the visibility of the surrounding tissue during the procedure as compared to a construction where slot 1034 is otherwise excluded (FIG. 10F shows increased visibility due to slot 1034).

In some examples, hook mechanism 1012 may be constructed independent of shaft 1010 and subsequently coupled (e.g., welded) thereto. In some such examples, the distal end of hook mechanism 1012 may include a bonding platform configured to be fixed to the distal end of shaft 1010. In the example shown in FIG. 10C, hook mechanism 1012 includes bonding platform 1038 in the shape of a partial cylinder that is bonded (e.g., via welding) to the exterior surface of shaft 1010, however other shapes and attachment mechanisms are also envisioned and shown and described elsewhere in this disclosure.

Hook mechanism 1012 may be constructed using any suitable materials including, for example, surgical grade stainless steel. Additional coatings or surface treatments may be applied to hook mechanism 1012 as desired.

FIG. 10F illustrates a schematic view of hook mechanism 1012 from the view point of an endoscope (e.g., 10320 AA Hopkins® Straight Forward Telescope) received within shaft 1010 such that the distal end of the endoscope is positioned approximately flush with the distal end of shaft 1010. The configuration of hook mechanism 1012 allows for the entire length of the central airway stent to be visually tracked by the clinician as the sidewall of the stent is introduced into slot 1024 of hook element 1022 and positioned against receiving platform 1020. As shown in FIG. 10F, both hook element 1022 and receiving platform 1020 may be constructed from as a continuous, solid member that provides a partially obstructed view of the surrounding tissue (open areas 1040 and 1042 represent spaces that would permit viewing of the surrounding patient tissue). In some examples, the visibility of the surrounding tissue may be improved by excluding the presence of retaining arm 1026, including one or more apertures within receiving platform 1020, including one or more apertures within hook element 1022, or a combination thereof.

FIGS. 11A-11G illustrate various view of another example central airway stent removal device 1100 that includes a mechanical, hook-capture removal mechanism that may offer enhanced surgical field visibility during stent retrieval. Central airway stent removal device 1100 includes an elongated shaft 1110, a hub assembly 1102 coupled to a proximal end of shaft 1110, and a hook mechanism 1112 coupled to a distal end of shaft 1110. Hub assembly 1102 and shaft 1110 may be substantially similar to hub assembly 1002 and shaft 1010 described above apart from any difference noted below. For example, shaft 1110 may by rigid or flexible and configured for use with either rigid of flexible endoscopes.

Hub assembly 1102 may include a grip 1114 and rotatable adaptor 1104. Grip 1114 (illustrated as a barrel grip) that can be grasped by a clinician and used to navigate hook mechanism 1112 to a target site and manipulate hook mechanism 1112 to engage or disengage with a central airway stent as needed. Adaptor 1104 may be configured to couple to an endoscope to secure central airway stent removal device 1100. The configuration of hub assembly 1102 allows for adaptor 1104 to be mechanically coupled to endoscope 1150 (FIG. 11F) while permitting grip 1114, shaft 1110, and hook mechanism 1112 rotate freely about the longitudinal axis of shaft 1110 relative to endoscope 1150. As described further below, having hook mechanism 1112 rotate relative to endoscope 1150 may aid in the removal of a central airway stent by allowing the stent to be rolled up on itself for easy extraction.

FIGS. 11B and 11C shows and example construction between grip 1114, rotatable adaptor 1104, and shaft 1110. As shown, rotatable adaptor 1104 may be constructed as a spool (e.g., cylinder with two discs on each side) with one end of the spool received within a complementary recess 1108 of grip 1114. Grip 1114 may be secured in place relative to shaft 1110 via locking device 1116 (e.g., collet or other suitable device) allowing rotatable adaptor 1104 to rotate freely within recess 1108.

In some examples, rotation of adaptor 1104 relative to grip 1114 may be unidirectional accomplished by including a ratchet mechanism (not shown) or other device within grip 1114. Having unidirectional rotation may be useful to inhibit spring-back by the central airway sent during removal. For example, as described further below, a central airway stent may be removed by engaging the stent with hook mechanism 1112 and rotating hook element 1122 relative to the stent to cause the stent to roll in on itself and collapse against hook mechanism 1112 so that it can be removed. In some examples, during the stent removal procedure, a clinician may rotate grip 1114 to collapse the central airway sent in on itself causing the stent to store up coiled spring force. The clinician may then inadvertently release grip 1114 causing the stent to release the stored coiled energy and rotate shaft 1110 in the opposite direction. Having grip 1114 rotate relative to adaptor 1104 in only one direction can prevent or reduce the amount of spring back that occurs.

As shown in FIGS. 11D and 11E, hook mechanism 1112 includes an elongated receiving platform 1120 that extends distally away from the distal end of shaft 1110 along the longitudinal axis of shaft 1110 and a hook element 1122 at the distal end of receiving platform 1120 that curls back toward hub assembly 1102 to define a receiving slot 1124 that opens towards the proximal end of device 1100 and is closed at the distal end. Hook mechanism 1112 also includes a mounting platform 1138 that secures hook mechanism 1112 to shaft 1110.

Hook mechanism 1112 defines at least one central channel 1126 that runs longitudinally along receiving platform 1120 to enhance surgical field visibility of hook mechanism 1112 when used in conjunction with endoscope 1150. FIG. 10G illustrates a schematic view of hook mechanism 1112 from the view point of an endoscope 1150 (e.g., 10320 AA Hopkins® Straight Forward Telescope) when received within shaft 1110 such that the distal end of the endoscope is positioned approximately flush with the distal end of shaft 1110. The presence of central channel 1126, along with the exclusion of retaining arm 1026, may enhance the visibility of the surrounding tissue. For example, compared to the visibility shown in FIG. 10F, both hook element 1122 and receiving platform 1120 provide an additional open viewing area 1142 due to central channel 1126 extending through both receiving platform 1120 and hook element 1122. In some examples, the enhanced visibility produced by open areas 1140 and 1142 of hook mechanism 1112 may provide greater than about 60%, greater than about 75%, or greater than about 85% tissue visibility from the perspective of endoscope 1150 (e.g., hook mechanism 1112 obstructs less than about 40%, less than about 25%, or less than about 15% of the view of the endoscope once it is coupled to device 1100).

In some examples, both receiving platform 1120 and hook element 1122 may be formed by a continuous, shaped wire that remains rigid during the stent retrieval process. Both ends of the shaped wire may be secured to mounting platform 1138. In some such examples, the middle of the wire will define the hooked-end 1128 of hook element 1122. Due to the rounded form of the wire, hooked end 1128 may naturally taper to produce a tapered surface at the entrance to slot 1124 that can help guide an end of the central airway stent into slot 1124. In some examples, mounting platform 1138 and the wire forming receiving platform 1120 and hook element 1122 may be forged as a single material. Hook mechanism 1112 may also provide an advantage over other designs by not having any moving parts (e.g., retaining arm 1026) thereby improving the simplicity of the construction and ease of use.

In some examples, receiving platform 1120 may define a recessed area 1130 configured to allow the proximal end of the central airway stent (e.g., the end of the stent opposite of that received within slot 1124) to seat within recessed area 1130. Recessed area 1130 may help to secure the stent against receiving platform 1120 during removal of the stent. In some examples, recessed area 1130 may be formed by having the wire forming receiving platform 1120 bend radially outward from the longitudinal axis of shaft 1110 relative to the plane bisecting the axis for a short length before bending back to extend substantially parallel (e.g., parallel or nearly parallel) to the longitudinal axis.

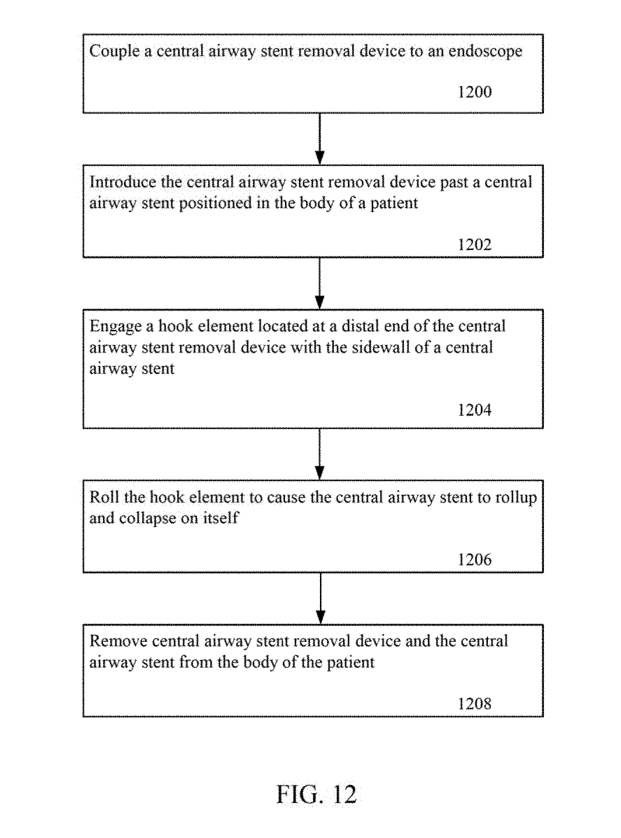

FIG. 12 is a flow diagram illustrating an example technique for using one of the central airway stent removal devices described herein. The below techniques are described in conjunction with central airway stent removal devices 1000 and 1100, however, the techniques may be used with other central airway stent removal devices described herein or central airway stent removal devices 1000 and 1100 may be used with other retrieval techniques. The technique of FIG. 12 is also described with respect to FIGS. 13A to 14B, which illustrate different ways in which hook mechanism 1112 engages and collapses central airway stent 1300.

The technique of FIG. 12 includes coupling central airway stent removal device 1100 to an endoscope (1200), introducing central airway stent removal device 1100 past a central airway stent positioned in the body of a patient (1202), engaging hook element 1122 located at a distal end of central airway stent removal device 1100 with the sidewall of a central airway stent (1204), rolling hook element 1122 to cause the central airway stent to rollup and collapse on itself (1206), and removing central airway stent removal device 1100 and the central airway stent from the body of the patient (1208).

As described above, central airway stent removal device 1100 may be configured to receive and couple with endoscope 1150. Shaft 1110 and hub assembly 1102 of central airway stent removal device 1100 may each define a central lumen 1106 configured to receive endoscope 1150 within a central lumen 1106. Hub assembly 1102 may include a rotatable adaptor 1104 configured to mechanically couple to endoscope 1150 to keep the two devices together while still allowing grip 1114, shaft 1110, and hook mechanism 1112 to rotated freely relative to the shaft of endoscope 1150.

Once coupled together, the distal end of endoscope 1150 and central airway stent removal device 1110 may be introduced and navigated through the airway to a patient to a treatment site containing a central airway stent 1300. Hook element 1112 located at the distal end of central airway stent removal device 1100 may be navigated past central airway stent 1300 (1202).

In some examples, hook element 1122 may be introduced through the inner lumen of central airway stent 1300 such that hook element 1122 at least partially past the distal end of the stent to allow hook element 1122, in particular slot 1124, to engage with the distal end 1302 and sidewall of central airway stent 1300 (1204). FIG. 14A illustrates this configuration with hook mechanism 1112 engaged with central airway stent 1300 such that the distal end 1302 of stent 1300 is received within slot 1124 defined by hook element 1122. In this configuration, receiving platform 1120 is positioned adjacent to the inner surface defining the inner lumen of stent 1300. In other examples, hook element 1122 may be inserted past central airway stent 1300 between the sidewall of the stent and the surrounding tissue as shown in FIG. 13A. In this configuration, receiving platform 1120 is positioned adjacent to the outer surface of stent 1300 and hook mechanism 1112 may help to disrupt and physically separate stent 1300 from the surrounding tissue if any tissue growth has occurred before removing stent 1300 from the body of the patient.

Once hook element 1122 has engaged with distal end 1302 of stent 1300 to received a portion of stent 1300 within slot 1124 (1204), retaining arm 1026 (if present) may be actuated to further secure stent 1300 against receiving platform 1020, 1120, and hook element 1122 may be rotated relative to stent 1300 about the longitudinal axis of shaft 1110 via grip 1114 (1206). The rotation of hook element 1122 may cause stent 1300 to at least partially rollup and collapse in on itself (FIGS. 13B and 14B) such that both hook mechanism 1112 and central airway stent 1300 may be withdrawn from the patient (1208) by, for example, retracting both components through bronchoscopy guide tube 1310.

Examples of the central airway stent removal device as disclosed herein provide a faster and less damaging approach to central airway stent positioning, removal, or repositioning. By having a device specifically intended for use with central airway stents, as are those devices disclosed herein, central airway stents are less likely to tear or degrade thus decreasing operating time and recovery time when compared with conventional forceps techniques.

Various examples of systems, devices, and methods have been described herein. These examples are given only by way of example and are not intended to limit the scope of the claimed inventions. It should be appreciated, moreover, that the various features of the examples that have been described may be combined in various ways to produce numerous additional examples. Moreover, while various materials, dimensions, shapes, configurations and locations, etc. have been described for use with disclosed examples, others besides those disclosed may be utilized without exceeding the scope of the claimed inventions.

Persons of ordinary skill in the relevant arts will recognize that the subject matter hereof may comprise fewer features than illustrated in any individual example described above. The examples described herein are not meant to be an exhaustive presentation of the ways in which the various features of the subject matter hereof may be combined. Accordingly, the examples are not mutually exclusive combinations of features; rather, the various examples can comprise a combination of different individual features selected from different individual examples, as understood by persons of ordinary skill in the art. Moreover, elements described with respect to one example can be implemented in other examples even when not described in such examples unless otherwise noted.

Although a dependent claim may refer in the claims to a specific combination with one or more other claims, other examples can also include a combination of the dependent claim with the subject matter of each other dependent claim or a combination of one or more features with other dependent or independent claims. Such combinations are proposed herein unless it is stated that a specific combination is not intended.

1. A central airway stent removal device for use under direct visual observation with a flexible or ridged endoscope, the device comprising:
an elongated shaft extending between a proximal end and a distal end; and
a hook mechanism attached to the distal end of the elongated shaft, wherein the hook mechanism comprises:
a receiving platform extending away from the elongated shaft; and
a hook element at the distal end of the hook mechanism, the hook element defining a receiving slot having an entrance that opens towards the elongated shaft, the receiving slot configured to receive an end of a stent intended to be removed from a body of a patient.

2. The central airway stent removal device of claim 1, further comprising a hub assembly connected to the proximal end of the elongated shaft, the elongated shaft and the hub assembly defining a central lumen extending there through configured to receive an endoscope, the hub assembly configured to mechanically couple to the endoscope.

3. The central airway stent removal device of claim 2, wherein the hub assembly comprises a grip and an adaptor, the adaptor being configured to receive and mechanically couple to the endoscope.

4. The central airway stent removal device of claim 3, wherein the grip is rotatable relative to the adaptor, and wherein the grip is connected to the elongated shaft such that rotation of the grip causes the shaft to rotate along a longitudinal axis relative to a shaft of the endoscope when the endoscope is coupled to the adaptor.

5. The central airway stent removal device of claim 4, wherein the grip is rotatable relative to the adaptor in only one rotational direction.

6. The central airway stent removal device of claim 4, wherein the grip comprises a ratchet mechanism configured to allow to the adaptor and the grip to rotate in only one rotational direction relative to each other.

7. The central airway stent removal device of claim 2, wherein the hook mechanism further comprises a retaining arm pivotably connected to the receiving platform, the retaining arm configured to transition from a open configuration where a portion of the stent can be received between the retaining arm and the receiving platform and a closed configuration where the portion of the stent is captured between the retaining arm and the receiving platform.

8. The central airway stent removal device of claim 7, further comprising at least one control wire extending between the hub assembly and the retaining arm along the elongated shaft, wherein longitudinal movement of the at least one control wire relative to the elongated shaft causes the retaining arm to transition between the open configuration and the closed configuration.

9. The central airway stent removal device of claim 1, wherein the receiving platform defines at least one channel that extends longitudinally along the receiving platform, wherein the at least one channel is configured to allow additional visibility of a treatment site from a perspective of the endoscope when the endoscope is coupled to the central airway stent removal device.

10. The central airway stent removal device of claim 1, wherein the hook mechanism comprises a shaped wire having both ends of the shaped wire secured to the elongated shaft, wherein the shaped wire forms both the receiving platform and the hook element.

11. The central airway stent removal device of claim 1, wherein the hook element comprises a tapered surface at the entrance to the receiving slot.

12. The central airway stent removal device of claim 1, wherein the hook mechanism obscures less than 40% of the surgical field from the perspective of the endoscope when the endoscope is coupled to the central airway stent removal device.

13. The central airway stent removal device of claim 1, wherein the hook mechanism obscures less than 25% of the surgical field from the perspective of the endoscope when the endoscope is coupled to the central airway stent removal device.

14. The central airway stent removal device of claim 1, wherein the receiving platform defines a recess that extends longitudinally along the receiving platform, the recess sized to receive an entire sidewall of a central airway stent within the recess.

15. The central airway stent removal device of claim 1, wherein the elongated shaft comprises a rigid tube defining a central lumen configured to receive an endoscope.

16. The central airway stent removal device of claim 15, wherein both the hook mechanism and the elongated shaft comprise one or more surgical grade metals.

17. A process for removing a central airway stent, the process comprising:
receiving an endoscope within a central lumen of a stent removal device, the stent removal device comprising:
an elongated shaft defining the central lumen configured to receive the endoscope; and
a hook mechanism attached to a distal end of the elongated shaft, wherein the hook mechanism comprises a receiving platform extending away from the elongated shaft, and a hook element at the distal end of the hook mechanism, the hook element defining a receiving slot having an entrance that opens towards the elongated shaft;
advancing the hook element into a body of a patient and past a central airway stent positioned within the body of the patient;
receiving a distal end of the central airway stent into the receiving slot such that a sidewall of the central airway stent is positioned adjacent to the receiving platform;
rotating the hook element relative to the central airway stent to cause the central airway stent to at least partially collapse inward; and
removing the central airway stent and the hook mechanism by proximally withdrawing the elongated shaft from the body of the patient.

18. The process of claim 17, further comprising actuating a retaining arm pivotably connected to the receiving platform, wherein actuating the retaining arm forces the central airway stent against the receiving platform.

19. The process of claim 17, wherein advancing the hook element into the body of the patient comprises introducing the hook mechanism and the elongated shaft through an inner lumen of a bronchoscopy guide tube.

20. A central airway stent removal device for use under direct visual observation with an endoscope, the device comprising:
a elongated shaft including an inner lumen arranged coaxially within the shaft, the inner lumen including a control wire;
a hook mechanism coupled to a distal end of the shaft, the hook mechanism comprising:
an inner support element including a hook detent arranged at a distal portion of the inner support element, the inner support element coupled to a distal end of the control wire, the inner support element and control wire configured to selectively and slidably move within the inner channel of the shaft via manipulation of the control wire; and
a receiving platform extending away from the elongated shaft; and
a hook element at the distal end of the hook mechanism, the hook element defining a receiving slot having an entrance that opens towards the elongated shaft,
wherein the receiving slot configured to receive an end of a stent, and wherein the hook detent is configured to selectively engage with the hook element to capture the stent between the receiving platform and the inner support element.
